(19) United States
(12) Patent Application Publication
(10) Pub. No.: US 2008/0248947 A1
(43) Pub. Date: Oct. 9, 2008

(54) Mixed metal oxide catalysts and catalytic processes for conversions of lower alkane hydrocarbons

(76) Inventors: Gerry W. Zajac, Warrenville, IL (US); Alakananda Bhattacharyya, Glen Ellyn, IL (US); Bhagya Chandra Sutradhar, Aurora, IL (US); James F. Brazdil, Glen Ellyn, IL (US)

Correspondence Address: INEOS USA LLC, 3030 WARRENVILLE RD, S/650, LISLE, IL, 60532, US

(21) Appl. No.: 11/732,213

(22) Filed: Apr. 3, 2007

(51) Int. Cl. B01J 23/16; C07C 253/24

(52) U.S. Cl. 502/312; 558/321

(57) ABSTRACT

Catalytic compositions and processes are disclosed for economical conversions of lower alkane hydrocarbons. Broadly, the present invention discloses solid promoter treated compositions containing mixed metal oxides that exhibit catalytic activity for ammoxidation of lower alkane hydrocarbons to produce an unsaturated nitrile in high yield. Generally, these solid oxide compositions comprise, as component elements, molybdenum (Mo), vanadium (V) niobium (Nb) and at least one active element selected from the group consisting of the elements having the ability to form positive ions. Mixed metal oxide catalytic compositions advantageously are formed process steps comprising impregnation of a base catalyst with an aqueous medium comprising sources of one or more promoter element drying the resulting material; and thereafter subjecting the dried material to heat treatment, under a gaseous atmosphere that is substantially free of dioxygen, at elevated temperatures of at least 400° C. Also described are methods for forming the improved catalysts having the desired crystalline structure and ammoxidation processes for conversion of lower alkanes.

### TECHNICAL FIELD

The present invention relates to solid compositions containing mixed metal oxides that exhibit catalytic activity for selective oxidation or ammoxidation of lower alkane hydrocarbons, such as propane and iso-butane, in the gaseous phase to produce oxygenate products, including unsaturated carboxylic acids and/or unsaturated mononitriles in high yield. The invention particularly relates to catalyst compositions, methods of preparing such catalyst compositions, and methods of using such catalyst compositions. More particularly, solid oxide compositions of the invention comprise, as component elements, molybdenum (Mo), vanadium (V) niobium (Nb) and at least one active element selected from the group consisting of the elements having the ability to form positive ions. Mixed metal oxide compositions of the invention advantageously are formed process steps comprising impregnation of a base catalyst with an aqueous medium comprising sources of at least one or more promoter elements, drying the resulting material; and thereafter subjecting the dried material to heat treatment, under a gaseous atmosphere that is substantially free of dioxygen, at elevated temperatures of at least 400° C. As will be described in greater detail hereinafter, the present invention provides methods for forming the improved catalysts of the invention and ammoxidation processes for conversion of lower alkanes.

### BACKGROUND OF THE INVENTION

Nitriles such as acrylonitrile and methacrylonitrile have long been industrially produced as important intermediates for the preparation of synthetic fibers, synthetic resins, synthetic rubbers and the like. A major use of acrylonitrile is in the form of fibers. Acrylonitrile-butadiene-styrene terpolymers (ABS) are important thermoplastic structural plastics. Nitrile-type rubbers, first commercialized as the German Buna-N type in 1930, are copolymers of acrylonitrile and a diene, usually butadiene.

The currently practiced commercial processes for the production of nitrites, such as acrylonitrile and methacrylonitrile, subject an alkene, i.e., propylene or isobutene, to reaction with ammonia and oxygen in the presence of a catalyst in a gas phase at a high temperature. Generally, the catalyst formulations employed are proprietary to the catalyst supplier, but the technology is well established. Furthermore, it is known to include additional starting materials, including additional reactants, such as molecular oxygen and/or steam, and inert materials, such as nitrogen and carbon dioxide, along with the hydrocarbon starting material.

Recently, in view of the relative abundance of lower alkanes relative to corresponding alkenes, resulting in price differences particularly between propane and propylene or between isobutane and isobutene, attention has been drawn to developing improved catalysts for producing nitrites from these, less expensive, lower alkanes. Propane or isobutane is used as starting material in a so-called ammoxidation reaction with ammonia and oxygen in a gas phase in the presence of a catalyst.

Catalysts containing molybdenum, vanadium, antimony and niobium which have been shown to be effective for conversion of propane to acrylonitrile and isobutane to methacrylonitrile (via an ammoxidation reaction) are described in numerous publications, patents and patent applications. See, for example, U.S. Pat. No. 5,750,760 to Ushikubo et al., U.S. Pat. No. 6,036,880 to Komada et al., U.S. Pat. No. 6,143,916 to Hinago et al., U.S. Pat. No. 6,514,902 to Inoue et al., U.S. Patent Application No. US 2003/0088118 A1 by Komadu et al., U.S. Patent Application No. 2004/0063990 A1 to Gaffney et al., U.S. Patent Application No. 2006/0167299 A1 to Gaffney et al., U.S. Patent Application No. 2006/0122055 A1 to Gaffney et al., U.S. Patent Application No. 2006/0183942 A1 to Gaffney et al., PCT Patent Application No. WO 2004/108278 A1 by Asahi Kasei Kabushiki Kaisha, Japanese Patent Application No. JP 1999/114426 A by Asahi Chemical Co., and Japanese Patent Application No. JP 2000/1126599 A by Asahi Chemical Co.

Oxide catalysts containing molybdenum, tellurium, vanadium and niobium are described in U.S. Pat. No. 5,049,692, U.S. Pat. No. 5,231,214, U.S. Pat. No. 5,281,745, U.S. Pat. No. 5,380,933, and U.S. Pat. No. 5,422,328. Further, oxide catalysts containing molybdenum, vanadium, niobium and antimony are described, for example, U.S. Pat. No. 4,760,159, U.S. Pat. No. 4,797,381, U.S. Pat. Appl. No. 2005/0054869 to Lugmair et al., and U.S. Pat. Appl. No. 2006/0122055 to Gaffney et al. However, none of these methods is fully satisfactory in the yield of the intended nitrites.

Although advancements have been made in the art in connection with catalysts containing molybdenum, vanadium, antimony and niobium effective for conversion of propane to acrylonitrile and isobutane to methacrylonitrile (via an ammoxidation reaction), the catalysts need further improvement before becoming commercially viable. In general, the art-known catalytic systems for such reactions suffer from generally low yields of the desired product.

Due to their extensive industrial uses, there is a continuing need for compositions having better catalytic activity for ammoxidation of lower alkane hydrocarbons to produce oxygenate products, including unsaturated carboxylic acids and/or unsaturated mononitriles in high yield It is an object of the invention to overcome one or more of the problems described above.

Other advantages of the invention will be apparent to those skilled in the art from a review of the following detailed description, taken in conjunction with the appended claims.

### SUMMARY OF THE INVENTION

In broad aspect, the present invention relates to improved catalyst compositions that exhibit an ability to facilitate selective oxidation or ammoxidation of a saturated hydrocarbon to the corresponding oxygenate products in high yield, and processes using these improved catalysts for economical conversions of lower alkane hydrocarbons to corresponding unsaturated carboxylic acids and/or unsaturated mononitriles. In particular, the present invention is directed to an improved catalyst and process for the ammoxidation of propane and/or isobutane to acrylonitrile and/or methacrylonitrile, respectively.

Accordingly, one aspect of the invention is a process for preparing solid compositions of the invention which comprises: (a) Providing a catalyst precursor, in dry particulate form, comprising elements, molybdenum (Mo), vanadium (V), at least one other element having the ability to form positive ions and to enhance the catalytic activity of the composition for the ammoxidation of propane and/or isobutane in the gaseous phase; (b) Calcining the catalyst precursor under conditions of calcination preselected such that at least one crystalline phase is formed in the resulting base catalyst which phase exhibits catalytic activity for ammoxidation or oxidation of propane and iso-butane in the gaseous phase; (c) Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and (d) Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

In another aspect of the invention the mixed metal oxide comprises as component elements, molybdenum (Mo), vanadium (V), at least one element selected from the group consisting of antimony (Sb) and tellurium (Te), and niobium (Nb). Particularly useful is mixed metal oxides which comprises at least a first crystalline phase that is characterized as having the M1 crystalline structure. In several embodiments of the invention, the mixed metal oxide further comprises a plurality of crystalline phases at least one of which is a mixed metal oxide comprising as component elements molybdenum (Mo), and antimony (Sb). In another embodiment the first phase is a mixed metal oxide comprising molybdenum (Mo), vanadium (V), antimony (Sb) and niobium (Nb).

In yet another aspect of the invention the aqueous medium comprising sources of one or more element selected from the group consisting of antimony (Sb) and tellurium (Te). Advantageously, the conditions of calcination comprise heating of the dry catalyst precursor, continuously or intermittently, from a preselected initial elevated temperature which is less than 400° C. to a 2^{nd} elevated temperature which is in the range of from 550° C. to 700° C.

In one embodiment of the invention the drying of material resulting from impregnation of the base catalyst with an aqueous medium utilizes a spray dryer means at elevated temperatures of from 150° C. to 300° C. measured at the entrance to the dryer section thereof. Beneficially, the resulting calcined base catalyst has a specific surface of from 5 m²/g to 30 m²/g.

Another aspect of the invention is a solid composition that exhibits improved catalytic activity for ammoxidation or oxidation of propane and iso-butane in the gaseous phase, which composition is formed by a process comprising: (i) Providing a catalyst precursor, in dry particulate form, which comprises sources of metal ions in amounts consistent with a nominal mixed oxide material represented by the empirical formula:

MoVₐSb_{b}Nb_{c}O_{δ}

where 0.1<a<1.0, 0.01<b<1.0, 0.001<c<0.25, and d is the number of oxygen atoms required to maintain elector-neutrality of the of the component elements present; (ii) Calcining the catalyst precursor under conditions of calcination preselected such that at least one crystalline phase is formed in the resulting base catalyst which phase exhibits catalytic activity for ammoxidation or oxidation of propane and iso-butane in the gaseous phase; (iii) Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and (iv) Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

Another aspect of the invention is a solid composition that exhibits improved catalytic activity for ammoxidation or oxidation of propane and iso-butane in the gaseous phase, which composition is formed by a process comprising:

Providing a catalyst precursor, in dry particulate form, which comprises sources of metal ions in amounts consistent with a nominal mixed oxide material represented by the empirical formula:

MoVₐSb_{b}Nb_{c}X_{d}A_{f}O_{δ}

where X is selected from the group consisting of Ti, Sn, Ge, Zr, Hf, and mixtures thereof,
A is selected from the group consisting of Ce, Nd and mixtures thereof,
0.1<a<0.8,
0.01<b<0.5,
0.001<c<0.1,
0.005<d<0.4,
0<f<0.1, and
δ is the number of oxygen atoms required to maintain Electro-neutrality of the other component elements present with the proviso that one or more of the other elements in the mixed oxide can be present in an oxidation state lower than its highest oxidation state;

Calcining the catalyst precursor under conditions of calcination preselected such that at least one crystalline phase is formed in the resulting base catalyst which phase exhibits catalytic activity for ammoxidation or oxidation of propane and iso-butane in the gaseous phase;

Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and

Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

Beneficially, in forming solid compositions of the invention, at least a portion of the catalyst precursor is formed by a process which comprises combining the sources of metal ions in aqueous solutions or aqueous mixtures, drying the resulting aqueous mixture to recover catalyst precursor, in dry particulate form. Advantageously, these aqueous mixtures are reacted at temperatures below about 100° C. and ambient, or near ambient, pressure.

Particularly useful conditions of calcination comprise heating of the dry catalyst precursor, continuously or intermittently, from a preselected initial elevated temperature which is in a range upward from 250° C. to 400° C. to a 2^{nd} elevated temperature which is in the range of from 550° C. to 700° C., and ambient, or near ambient, pressure.

In yet another aspect of the invention, the solid composition is formed by a process comprising:

Providing a catalyst precursor, in dry particulate form, which comprises sources of metal ions in amounts consistent with a nominal mixed oxide material represented by the empirical formula:

MoVₐSb_{b}Nb_{c}Ti_{d}O_{δ}

where 0.1<a<1.0,
0.01<b<1.0,
0.01<c<0.25,
0.005<d<0.4, and
δ is the number of oxygen atoms required to maintain electro-neutrality of the other component elements present;

Calcining the catalyst precursor under conditions of calcination preselected such that at least one crystalline phase is formed in the resulting base catalyst which phase exhibits catalytic activity for ammoxidation or oxidation of propane and iso-butane in the gaseous phase;

Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and

Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

The invention also provides a process for catalytic conversion of propane or isobutane to an unsaturated nitrile or unsaturated carboxylic acid by ammoxidation in the presence of ammonia and a source of dioxygen or oxidation in the presence of a source of dioxygen, respectively, using a particulate solid catalyst which comprises one or more mixed metal crystalline oxide composition according to any aspect of the invention.

For a more complete understanding of the present invention, reference should now be made to the embodiments described in greater detail below and by way of examples of the invention.

### GENERAL DESCRIPTION

The catalyst of the present invention may be used either supported or unsupported (i.e. the catalyst may comprise a support). Suitable supports are silica, alumina, zirconia, titania, or mixtures thereof. However, when zirconia or titania are used as support materials then the ratio of molybdenum to zirconium or titanium increases over the values shown in the above formulas, such that the Mo to Zr or Ti ratio is between about 1 to 10. A support typically serves as a binder for the catalyst resulting in a harder and more attrition resistant catalyst. However, for commercial applications, an appropriate blend of both the active phase (i e. the complex of catalytic oxides described above) and the support is helpful to obtain an acceptable activity and hardness (attrition resistance) for the catalyst. Directionally, any increase in the active phase decreases the hardness of the catalyst. The support comprises between 10 and 90 weight percent of the supported catalyst. Typically, the support comprises between 40 and 60 weight percent of the supported catalyst. In one embodiment of this invention, the support may comprise as little as about 10 weight percent of the supported catalyst. In one embodiment of this invention, the support may comprise as little as about 30 weight percent of the supported catalyst. In another embodiment of this invention, the support may comprise as much as about 70 weight percent of the supported catalyst. Support materials are available which may contain one or more promoter elements, and such promoter elements may be incorporated into the catalyst via the support material.

The invention contemplates continuous processes for recovery and purification of organic values from hot gaseous mixtures which are obtained by catalytic ammoxidation of a light alkane hydrocarbon compounds. More particularly, this invention relates to recovery and refining of valuable nitrogen-containing organic compounds formed by catalytic oxidation of least one feed compound selected from the group consisting of propane and isobutane in the presence of ammonia to produce a gaseous reactor effluent containing the corresponding unsaturated mononitrile.

Propane is preferably converted to acrylonitrile and isobutane to methacrylonitrile, by providing one or more of the aforementioned catalysts in a gas-phase flow reactor, and contacting the catalyst with propane or isobutane in the presence of oxygen (e.g. provided to the reaction zone in a feedstream comprising an oxygen-containing gas, such as and typically air) and ammonia under reaction conditions effective to form acrylonitrile or methacrylonitrile. For this reaction, the feed stream preferably comprises propane or isobutane, an oxygen-containing gas such as air, and ammonia with the following molar ratios of propane or isobutane to oxygen in a ratio ranging from about 0.125 to about 5, and preferably from about 0.25 to about 2.5, and propane or isobutane to ammonia in a ratio ranging from about 0.3 to about 2.5, and preferably from about 0.5 to about 2.0. The feed stream can also comprise one or more additional feed components, including acrylonitrile or methacrylonitrile product (e.g., from a recycle stream or from an earlier-stage of a multi-stage reactor), and/or steam. For example, the feedstream can comprise about 5 percent to about 30 percent by weight relative to the total amount of the feed stream, or by mole relative to the amount of propane or isobutane in the feed stream. In one embodiment the catalyst compositions described herein are employed in the ammoxidation of propane to acrylonitrile is a once-through process, i.e., it operates without recycle of recovered but unreacted feed materials.

The specific design of the gas-phase flow reactor is not narrowly critical. Hence, the gas-phase flow reactor can be a fixed-bed reactor, a fluidized-bed reactor, or another type of reactor. The reactor can be a single reactor, or can be one reactor in a multi-stage reactor system. Preferably, the reactor comprises one or more feed inlets for feeding a reactant feedstream to a reaction zone of the reactor, a reaction zone comprising the mixed metal oxide catalyst, and an outlet for discharging reaction products and unreacted reactants.

The reaction conditions are controlled to be effective for converting the propane to acrylonitrile, respectively, or the isobutane to methacrylonitrile. Generally, reaction conditions include a temperature ranging from about 300° C. to about 550° C., preferably from about 325° C. to about 500° C., and in some embodiments from about 350° C. to about 450° C., and in other embodiments from about 430° C. to about 520° C. Generally, the flow rate of the propane or isobutene containing feedstream through the reaction zone of the gas-phase flow reactor can be controlled to provide a weight hourly space velocity (WHSV) ranging from about 0.02 to about 5, preferably from about 0.05 to about 1, and in some embodiments from about 0.1 to about 0.5, in each case, for example, in grams propane or isobutane to grams of catalyst. The pressure of the reaction zone can be controlled to range from about 0 psig to about 200 psig, preferably from about 0 psig to about 100 psig, and in some embodiments from about 0 psig to about 50 psig.

The resulting acrylonitrile methacrylonitrile product can be isolated, if desired, from other side-products and/or from unreacted reactants according to methods known in the art.

The catalyst compositions described herein when employed in the single pass (i.e. no recycle) ammoxidation of propane are capable of producing a yield of about 57-58 percent acrylonitrile, with a selectivity of about 24 percent to COₓ (carbon dioxide+carbon monoxide), and a selectivity of about 13 percent to a mixture of hydrogen cyanide (HCN) and acetonitrile or methyl cyanide (CH₃CN). The effluent of the reactor may also include unreacted oxygen (O₂), ammonia (NH₃) and entrained catalyst fines.

Processes for recovery and purification of the reaction products include quenching the gaseous reactor effluent with an aqueous quench liquid; forming an aqueous solution comprising the corresponding unsaturated mononitrile, hydrogen cyanide and other organic co-products; and using an integrated sequence of distillations and phase separations to recover for recycle of a useful aqueous liquid, and obtain valuable nitrogen-containing organic compounds and hydrogen cyanide products.

Propane, ammonia and oxygen mix together in the reactor and oxidation of propylene in the presence of ammonia takes place on the surface of the fluidized catalyst. A set of complex exothermic reactions takes place, thereby forming the following products: acrylonitrile, hydrogen cyanide, carbon dioxide, carbon monoxide, acetonitrile, acrolein, acrylic acid, water, other higher nitrites, aldehydes, ketones, acetic acid and a number of miscellaneous unknown organic compounds. Conversions of the three feeds are less than 100 percent, thus unreacted propane, ammonia, oxygen and nitrogen are contained in the reactor effluent gas. The source of propane typically contains a small amount of propylene and some heavier hydrocarbon compounds most of which are purged from the process unreacted. A portion of the heat of the exothermic reaction is removed by sets of steam coils which generate and superheat waste steam at approximately 600 psig for process uses such as heat input for distillations in the products recovery and purification section of the process. Reactor effluent gas passes through cyclones, which remove catalyst fines from the gas. The gas is then further cooled in a reactor effluent cooler, which is comprised of a shell and tube exchanger using boiler feed-water as the cooling source.

As is well known, performance of the oxidation catalysts is an important factor, perhaps the most significant factor, in the economics of this and other oxidation processes. Catalyst performance is measured by activity, i.e., conversion of reactants, selectivity, i.e. conversion of reactant to desired product, rate of production of desired product per unit of reactor volume per unit of time, and catalyst life, i.e. effective time on-stream before significant loss of activity or selectivity.

Factors upon which catalyst performance depends include composition, the methods of preparation, support, and calcination conditions. In addition to chemical performance requirements, other key properties include surface area, porosity, density, pore size distribution, hardness, strength, and resistance to mechanical attrition, particularly for fluid bed catalysts.

Typically, the ammoxidation process is carried out in a fluid-bed reactor. Where high alkane conversions are obtained, a single pass system is satisfactory with a residence time of a few seconds is typical. Commercially recoverable quantities of acetonitrile and hydrocyanic acid are optional co-products. Approximately stoichiometric quantities of propane, ammonia, and dioxygen are introduced into a fluidized bed of catalytic particles. Suitable operating conditions include pressures in a range from about 18 to about 50 psia (about 126 to about 350 kPa), more preferably from about 20 to about 40 psia (about 140 to about 280 kPa). Generally, temperatures are in a range from about 700° to 1000° F. (371° to 538° C.), preferable in a range from about 750° to 950° F. (399° to 510° C.). Heat of reaction is removed by generation of steam to control the temperature and generating steam at temperatures of from about 300° to about 500° C. elevated pressure.

### EXAMPLES OF THE INVENTION

The following examples will serve to illustrate certain specific embodiments of the inventions herein disclosed. These Examples should not, however, be construed as limiting the scope of the novel invention as there are many variations which may be made thereon without departing from the spirit of the disclosed invention, as those of skill in the art will recognize.

### General

In order to illustrate the instant invention, samples of a base catalyst, with and without various catalyst modifiers, were prepared and then evaluated under similar reaction conditions. The compositions listed below are nominal compositions, based on the total metals added in the catalyst preparation. Since some metals may be lost or may not completely react during the catalyst preparation, the actual composition of the finished catalyst may vary slightly from the nominal compositions shown below.

### Drying Step

The aqueous mixture of ingredients is dried to thereby provide a dry catalyst precursor. Drying may be conducted by conventional methods, such as spray drying or evaporation drying. Spray drying is particularly useful, because a fine, spherical, dry catalyst precursor is obtained. The spray drying can be conducted by centrifugation, by the two-phase flow nozzle method or by the high-pressure nozzle method. As a heat source for drying, it is preferred to use air that has been heated by steam, an electric heater and the like. It is preferred that the temperature of the spray dryer at an entrance to the dryer section thereof is from 150° C. to 300° C.

### Calcination Step

In the calcination step, the dry catalyst precursor is converted into a mixed metal oxide catalyst. Calcinations can be conducted using a rotary kiln, a fluidized-bed kiln or the like. When calcination of the dry catalyst precursor is conducted in a stationary state, problems possibly arise in that the precursor cannot be evenly calcined, thus leading to a deterioration of the properties of the catalyst obtained and also to a breakage or cracking of the catalyst obtained.

Conditions of calcination preselected such that the catalyst formed has a specific surface are of from 5 m²/g to 30 m²/g. Typically, calcination is conducted under calcination conditions wherein the heating temperature of the dry catalyst precursor is continuously or intermittently elevated from a temperature which is less than 400° C. to a temperature which is in the range of from 550° C. to 700° C. The calcination can be conducted in air or under a flow of air. However, at least a part of the calcination is preferably conducted in an atmosphere of an inert gas (e.g., under a flow of an inert gas), such as nitrogen gas that is substantially free of dioxygen.

### Catalyst Testing

Catalyst was evaluated in a 40 cc fluid bed reactor having a diameter of 1-inch. The reactor was charged with about 20 to 45 g of particulate catalyst. Propane was fed into the reactor at a rate of about 0.05 to 0.15 WWH (i.e., weight of propane/weight of catalyst/hour). Ammonia was fed into the reactor at a flow rate such that ammonia to propane ratio was in the range for about 1 to 1.5. Pressure inside the reactor was maintained at about 2 to 15 psig. Reaction temperatures were in the range of about 420° C. to 460° C.

### Example 1

This example illustrates preparation of a mixed metal oxide catalyst having a nominal composition represented by

Mo V_{0.21}Sb_{0.24}Nb_{0.09 O}_{δ}

### (Preparation of Dried Catalyst Precursor)

A solution was prepared by mixing 230 g ammonium heptamolybdate and 1160 g water. To this solution was added 32 g ammonium metavanadate and 38 g of antimony (III) oxide (Sb₂O₃). The mixture was heated to 90° C. and stirred for 2.5 hours. The solution was then cooled to 70° C. and 490 g of a Nalco silica sol (96SN036, 30.6 solids) and 44.6 g of 30 percent H₂O₂ was added. The solution was stirred for 1 hour at 50° C. (Mixture 1).

Another mixture was prepared by adding 7.65 g of antimony (III) oxide (Sb₂O₃), to 181.33 g of a 0.65 molar (in Nb) niobic acid/oxalic acid solution, previously prepared. To this mixture were added 38.6 g of 30 percent H₂O₂. The mixture was stirred for 30 min at room temperature (Mixture 2).

Mixture 2 was added to Mixture 1 while stirring. A solution prepared by mixing 756 g fumed silica and 1125 g water was added to the combination of mixtures, stirred for 15 min and spray dried. The resulting dried catalyst precursor was identified as dried four components catalyst Precursor A.

(Two step calcination of catalyst precursor)

About 75 g of Precursor A was calcined under nitrogen in a 1 foot vertical tube in two steps. After raising the temperature of the loaded 1 foot vertical tube at the rate of about 1.2° C./min, to 345° C., the temperature was maintained at 345° C. for 4 hours. In the 2^{nd} step the temperature was further raised at the rate of about 2.3° C./min to a temperature of 640° C. After dwelling for 2 hours at 640° C., the calcination was completed. This catalyst was evaluated in a 40 cc fluid bed reactor.

### Example 2

This example illustrates preparation of a mixed metal oxide catalyst according to the invention by impregnation of a four component base material with a predetermined amount of source of tellurium.

Fifty grams of a spray dried 4-metal components catalyst precursor, prepared as described in Example 1 for Precursor A, was calcined in a two step process under nitrogen with an intermediate temperature of 345° C. and final temperature of 630° C. 1 gram of the calcined material was weighed into a crucible and put into a 600° C. furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 46 g of calcined catalyst (44.9 g dry basis) was weighed out in a beaker. 1.089 g of Te(OH)₆ was added to 13.24 mL of water (calculated from pore volume test) to obtain a solution. This solution was then added to the catalyst with stirring until the catalyst reached a point of incipient wetness producing a catalyst with 0.04 moles of tellurium per mole of molybdenum. The catalyst was placed in a 90° C. oven overnight to dry in air. The material was then subjected to heat treatment under nitrogen at 450° C. for 2 hours and the resulting material was identified as Catalyst 4+0.04Te.

### Example 3

This example illustrates preparation of a mixed metal oxide catalyst according to the invention by impregnation prior to calcination of a four component base material with a predetermined amount of source of tellurium.

1 gram of spray dried 4-metal components catalyst precursor, prepared as described in Example 1 for Precursor A, was weighed into a crucible and put into a 600° C. furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 72.4 g of catalyst (60 g when account for the moisture content) was weighed out in a beaker. 1.46 g of Te(OH)₆ was added to 35 mL of water (calculated from pore volume test) to obtain a solution. This solution was then added to the catalyst precursor with stirring until the solids reached a point of incipient wetness. The impregnated base material was placed in a 90° C. oven overnight to dry in air. A two step calcination of the dry material, under nitrogen, used an initial temperature of 345° C. and final temperature of 630° C. to producing a catalyst with 0.04 moles of tellurium per mole of molybdenum that was identified as Catalyst uncal 4+0.04Te.

### Example 4

This example illustrates preparation of a mixed metal oxide catalyst according to the invention by impregnation prior to calcination of a four component base material with another predetermined amount of source of tellurium.

1 gram of a spray dried 4-metal components catalyst precursor, prepared as described in Example 1 for Precursor A, was weighed into a crucible and put into a 600° C. furnace for 3 hours, cooled down and weighed. The moisture content of the material was then determined from the weight loss. To determine the pore volume, 0.5 g of material was added to a beaker, and distilled water from a burette was added very slowly with stirring until the solids reached incipient wetness. The pore volume per gram of catalyst precursor was then determined. 72.4 g of catalyst precursor (60 g when account for the moisture content) was weighed out in a beaker. 1.46 g of Te(OH)₆ was added to 35 mL of water (calculated from pore volume test) to obtain a solution. This solution was then added to the catalyst precursor with stirring until the solids reached a point of incipient wetness.

A two step calcination of the dry material, under nitrogen, used an initial temperature of 345° C. and final temperature of 630° C. to producing a catalyst with 0.06 moles of tellurium per mole of molybdenum that was identified as Catalyst uncal 4+0.06Te.

### Example 5

This example illustrates preparation of a mixed metal oxide catalyst according to the invention by impregnation of a six component base material with a predetermined amount of source of tellurium.

50 grams of a spray dried six components catalyst precursor, prepared as described in Example 11 for Precursor B, (Mo, V, Sb, Nb, Ti, and Ce) was calcined in a two step process under nitrogen with an intermediate temperature of 345° C. and final temperature of 630° C. 1 gram of the calcined material was weighed into a crucible and put into a 600 furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 49.44 g of calcined catalyst (48.25 g when account for the moisture content) was weighed out in a beaker. 1.121 g of Te(OH)₆ was added to 7.12 mL of water (calculated from pore volume test) to obtain a solution. This solution was then added to the catalyst with stirring until the catalyst reached a point of incipient wetness producing a catalyst with 0.04 moles of tellurium per mole of molybdenum. The catalyst was placed in a 90° C. oven overnight to dry in air. The material was then subjected to heat treatment under nitrogen at 450° C. for 2 hours, and the resulting material was identified as Catalyst 6+0.04Te.

### Example 6

This example illustrates preparation of a mixed metal oxide catalyst according to the invention by impregnation of a six component base material with another predetermined amount of source of tellurium.

50 grams of a spray dried six components catalyst precursor, prepared as described in Example 11 for Precursor B, (Mo, V, Sb, Nb, Ti, and Ce) was calcined in a two step process under nitrogen with an intermediate temperature of 345° C. and final temperature of 630° C. 1 gram of the calcined material was weighed into a crucible and put into a 600° C. furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 48.12 g of calcined catalyst (46.97 g when account for the moisture content) was weighed out in a beaker. 1.637 g of Te(OH)₆ was added to 6.93 mL of water (calculated from pore volume test) to obtain a solution. This solution was then added to the catalyst with stirring until the catalyst reached a point of incipient wetness producing a catalyst with 0.06 moles of tellurium per mole of molybdenum. The catalyst was placed in a 90° C. oven overnight to dry in air. The material was then subjected to heat treatment under nitrogen at 450° C. for 2 hours, and the resulting material was identified as Catalyst 6+0.06Te.

### Example 7

This example illustrates preparation of a mixed metal oxide catalyst according to the invention by impregnation of a six component base material with another predetermined amount of source of tellurium.

50 grams of a spray dried six components catalyst precursor, prepared as described in Example 11 for Precursor B, (Mo, V, Sb, Nb, Ti, and Ce) was calcined in a two step process under nitrogen with an intermediate temperature of 345° C. and final temperature of 630° C. 1 gram of the calcined material was weighed into a crucible and put into a 600 furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 49.01 g of calcined catalyst (47.83 g when account for the moisture content) was weighed out in a beaker. 0.834 g of Te(OH)₆ was added to 7.65 mL of water (calculated from pore volume test) to obtain a solution. This solution was then added to the catalyst with stirring until the catalyst reached a point of incipient wetness producing a catalyst with 0.03 moles of tellurium per mole of molybdenum. The catalyst was placed in a 90° C. oven overnight to dry in air. The material was then subjected to heat treatment under nitrogen at 450° C. for 2 hours, and the resulting material was identified as Catalyst 6+0.03Te.

### Example 8

This example illustrates preparation of a mixed metal oxide catalyst according to the invention by impregnation of a six component base material with yet another predetermined amount of source of tellurium.

50 grams of a spray dried six components catalyst precursor, prepared as described in Example 11 for Precursor B, (Mo, V, Sb, Nb, Ti, and Ce) was calcined in a two step process under nitrogen with an intermediate temperature of 345° C. and final temperature of 630° C. 1 gram of the calcined material was weighed into a crucible and put into a 600° C. furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness The pore volume per gram of catalyst could then be determined. 49.98 g of calcined catalyst (47.80 g when account for the moisture content) was weighed out in a beaker. 0.555 g of Te(OH)₆ was added to 7.64 mL of water (calculated from pore volume test) to obtain a solution. This solution was then added to the catalyst with stirring until the catalyst reached a point of incipient wetness producing a catalyst with 0.02 moles of tellurium per mole of molybdenum. The catalyst was placed in a 90° C. oven overnight to dry in air. The material was then subjected to heat treatment under nitrogen at 450° C. for 2 hours, and the resulting material was identified as Catalyst 6+0.02Te.

### Example 9

This example illustrates preparation of a mixed metal oxide catalyst according to the invention by impregnation of a six component base material with predetermined amounts of a source of tellurium and a source of antimony.

50 grams of a spray dried six components catalyst precursor, prepared as described in Example 11 for Precursor B, (Mo, V, Sb, Nb, Ti, and Ce) was calcined in a two step process under nitrogen with an intermediate temperature of 345° C. and final temperature of 630° C. 1 gram of the calcined material was weighed into a crucible and put into a 600 furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 44.5 g of calcined catalyst (43.43 g when account for the moisture content) was weighed out in a beaker. 11.43 g of antimony Sol (15.5 percent Sb₂O₅ by weight) was added to the catalyst with stirring until the catalyst reached a point of incipient wetness producing a catalyst with an additional 0.1 moles of antimony per mole of molybdenum. This material was dried in a 90° C. oven overnight. 1.01 g of Te(OH)₆ was added to 6.78 mL of water (calculated from pore volume test) to obtain a solution. This solution was then added to the dried catalyst with stirring until the catalyst reached a point of incipient wetness producing a catalyst with 0.04 moles of tellurium per mole of molybdenum. The catalyst was placed in a 90° C. oven overnight to dry in air. The material was then subjected to heat treatment under nitrogen at 450° C. for 2 hours, and the resulting material was identified as Catalyst 6+0.04Te+0.1Sb.

### Example 10

This example illustrates preparation of a mixed metal oxide catalyst according to the invention by impregnation of a six component base material with a predetermined amount a source of antimony.

50 grams of a spray dried six components catalyst precursor, prepared as described in Example 11 for Precursor B, (Mo, V, Sb, Nb, Ti, and Ce) was calcined in a two step process under nitrogen with an inital temperature of 345° C. and final temperature of 630° C. 1 gram of the calcined material was weighed into a crucible and put into a 600° C. furnace for 3 hours, cooled down and weighed. The moisture content of the material was then determined from the weight loss. To determine the pore volume, 0.5 g of material was added to a beaker, and distilled water from a burette was added very slowly with stirring until catalyst reached incipient wetness. The pore volume per gram of catalyst could then be determined. 44.5 g of calcined catalyst (43.43 g when account for the moisture content) was weighed out in a beaker. 11.47 g of antimony Sol (15.5 percent Sb₂O₅ by weight) was added to the catalyst with stirring until the catalyst reached a point of incipient wetness producing a catalyst with an additional 0.1 moles of tellurium per mole of molybdenum. The catalyst was placed in a 90° C. oven overnight to dry in air. The material was then subjected to heat treatment under nitrogen at 450° C. for 2 hours, and the resulting material was identified as Catalyst 6+0.1Sb.

### Example 11

This example illustrates preparation of a mixed metal oxide catalyst having a nominal composition represented by

Mo V_{0.3}Sb_{0.2}Nb_{0.08}Ti_{0.1}Ce_{0.005}O_{δ}

(Preparation of dried catalyst precursor)

A solution was prepared by mixing 222.4 g ammonium heptamolybdate and 1160 g water. To this solution was added 44.22 g ammonium metavanadate and 30.67 g of Sb₂ O₃. The mixture was heated to 90° C. and stirred for 2.5 h. The solution was then cooled to 70° C. and 466.9 g of a Nalco silica sol (96SN036, 32 percent solids) and 44.5 g of 30 percent H₂O₂ was added. The solution was stirred for 1 h at 50° C. (Mixture 1)

A mixture was prepared by adding 6.1 g of Sb₂ O₃ to a 153.7 g of a 0.63 molar (in Nb) niobic acid/oxalic acid solution, previously prepared. To this mixture were added 38.5 g of 30 percent H₂O₂, 2.07 g Ce(acetate)₃ 1.5 H₂O, and 10.07 g of TiO₂. The mixture was stirred for 30 min at room temperature (Mixture 2).

Mixture 2 was added to Mixture 1 while stirring. To this was added a solution prepared by mixing 74.7 g fumed silica. and 1125 g water. The final mixture was stirred for 15 min and spray dried. The resulting dried catalyst precursor was identified as dried six components catalyst Precursor B.

(Two step calcination of catalyst precursor)

About 75 g of this dried material was calcined under nitrogen in a 1 foot vertical tube in two steps. After raising the temperature of the loaded 1 foot vertical tube at the rate of about 1.2° C./min, to 345° C., the temperature was maintained at 345° C. for 4 hours. In the 2^{nd} step the temperature was again raised at the rate of about 2.3° C./min to a temperature of 600° C. After dwelling for 2 hours at 600° C., the calcination was completed. This catalyst was evaluated in a 40 cc fluid bed reactor.

TABLE
Performance of Te and Sb promoted 4-component and 6-component
base materials.

| Ex. | Run | TIS | C3 Conv, | AN Sel, | AN, | | |
|---|---|---|---|---|---|---|---|
| No. | ° C. | h | % | % | % | WWH | Catalyst |
| 1 | 440 | 27 | 82.3 | 41.2 | 33.9 | 0.05 | Base 4 comp |
| | 440 | 48 | 75.1 | 43.4 | 32.6 | 0.07 | |
| 2 | 440 | 65 | 78.8 | 51.2 | 40.3 | 0.049 | 4 + 0.04Te |
| | 450 | 218 | 80.6 | 53.8 | 43.4 | 0.07 | |
| 3 | 440 | 47 | 83.3 | 39.7 | 33.1 | 0.05 | Uncal 4 + |
| | 440 | 234 | 78.8 | 32.6 | 25.7 | 0.05 | 0.04Te |
| 4 | 430 | 3.8 | 58.7 | 45.3 | 26.6 | 0.049 | Uncal 4 + |
| | | | | | | | 0.06Te |
| 5 | 440 | 138 | 81.7 | 48.9 | 40.0 | 0.05 | 6 + 0.04Te |
| | 455 | 332 | 78.3 | 48.3 | 37.8 | 0.07 | |
| 6 | 440 | 21 | 58.1 | 34.5 | 20.0 | 0.05 | 6 + 0.06Te |
| 7 | 440 | 23.3 | 73.6 | 46.6 | 34.3 | 0.049 | 6 + 0.03Te |
| | 440 | 45 | 65.4 | 49.9 | 32.6 | 0.069 | |
| 8 | 440 | 67 | 79.2 | 41.3 | 32.7 | 0.05 | 6 + 0.02Te |
| 9 | 440 | 67 | 75.9 | 51.6 | 39.2 | 0.049 | 6 + 0.04Te + |
| | 450 | 214 | 79.4 | 50.6 | 40.0 | 0.049 | 0.1Sb |
| 10 | 440 | 19 | 89.6 | 47.7 | 42.8 | 0.049 | 6 + 0.1Sb |
| | 450 | 24 | 90.5 | 47.6 | 43.1 | 0.049 | |
| | 450 | 184 | 90.3 | 45.3 | 40.9 | 0.06 | |
| 11 | 440 | 66 | 82 | 41.4 | 34.0 | 0.05 | Base 6 comp |

For the purposes of the present invention, “predominantly” is defined as more than about fifty percent. “Substantially” is defined as occurring with sufficient frequency or being present in such proportions as to measurably affect macroscopic properties of an associated compound or system. Where the frequency or proportion for such impact is not clear, substantially is to be regarded as about twenty per cent or more. The term “a feedstock consisting essentially of” is defined as at least 95 percent of the feedstock by volume. The term “essentially free of” is defined as absolutely except that small variations which have no more than a negligible effect on macroscopic qualities and final outcome are permitted, typically up to about one percent.

That which is claimed is:

1. A solid composition that exhibits improved catalytic activity for ammoxidation or oxidation of propane and iso-butane in the gaseous phase, which composition is formed by a process comprising:
Providing a catalyst precursor, in dry particulate form, comprising elements, molybdenum (Mo), vanadium (V), at least one other element having the ability to form positive ions and to enhance the catalytic activity of the composition for the ammoxidation of propane and/or isobutane in the gaseous phase;
Calcining the catalyst precursor under conditions of calcination preselected such that at least one crystalline phase is formed in the resulting base catalyst which phase exhibits catalytic activity for ammoxidation or oxidation of propane and iso-butane in the gaseous phase;
Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and
Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

2. The composition of claim 1 wherein the mixed metal oxide comprises as component elements, molybdenum (Mo), vanadium (V), at least one element selected from the group consisting of antimony (Sb) and tellurium (Te), and niobium (Nb).

3. The composition of claim 1 wherein the mixed metal oxide comprises at least a first crystalline phase that is characterized as having a M1 crystalline structure.

4. The composition of claim 3 wherein the first phase is a mixed metal oxide comprising molybdenum (Mo), vanadium (V) and niobium (Nb), and antimony (Sb) or tellurium (Te).

5. The composition of claim 1 wherein the aqueous medium comprising sources of one or more element is selected from the group consisting of antimony (Sb) and tellurium (Te).

6. The composition of claim 1 wherein the conditions of calcination comprise heating of the dry catalyst precursor, continuously or intermittently, from a preselected initial elevated temperature which is less than 400° C. to a 2^{nd} elevated temperature which is in the range of from 550° C. to 700° C.

8. The composition of claim 7 wherein the drying of material resulting from impregnation of the base catalyst with an aqueous medium utilizes a spray dryer means at elevated temperatures of from 150° C. to 300° C. measured at the entrance to the dryer section thereof.

9. The composition of claim 7 wherein the resulting calcined base catalyst has a specific surface of from 5 m²/g to 30 m²/g.

10. The composition of claim 1 wherein the mixed metal oxide further comprises a plurality of crystalline phases at least one of which is a mixed metal oxide comprising as component elements molybdenum (Mo), and antimony (Sb).

11. A solid composition that exhibits improved catalytic activity for ammoxidation or oxidation of propane and iso-butane in the gaseous phase, which composition is formed by a process comprising:
Providing a catalyst precursor, in dry particulate form, which comprises sources of metal ions in amounts consistent with a nominal mixed oxide material represented by the empirical formula:
MOVₐSb_{b}Nb_{c}O_{δ}
where 0.1<a<1.0,
0.01<b<1.0,
0.001<c<0.25, and
δ is the number of oxygen atoms required to maintain electro-neutrality of the other component elements present;
Calcining the catalyst precursor under conditions of calcination preselected such that at least one crystalline phase is formed in the resulting base catalyst which phase exhibits catalytic activity for ammoxidation or oxidation of propane and iso-butane in the gaseous phase;
Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and
Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

12. A solid composition that. exhibits improved catalytic activity for ammoxidation or oxidation of propane and iso-butane in the gaseous phase, which composition is formed by a process comprising:
Providing a catalyst precursor, in dry particulate form, which comprises sources of metal ions in amounts consistent with a nominal mixed oxide material represented by the empirical formula:
MoVₐSb_{b}Nb_{c}X_{d}A_{f}O_{δ}
where X is selected from the group consisting of Ti, Sn, Ge, Zr, Hf, and mixtures thereof,
A is selected from the group consisting of Ce, Nd and mixtures thereof,
0.1<a<0.8,
0.01<b<0.5,
0.001<c<0.1,
0.005<d<0.4,
0<f<0.1, and
δ is the number of oxygen atoms required to maintain electro-neutrality of the other component elements present with the proviso that one or more of the other elements in the mixed oxide can be present in an oxidation state lower than its highest oxidation state;
Calcining the catalyst precursor under conditions of calcination preselected such that at least one crystalline phase is formed in the resulting base catalyst which phase exhibits catalytic activity for ammoxidation or oxidation of propane and iso-butane in the gaseous phase;
Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and
Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

13. The solid composition of claim 12 wherein at least a portion of the catalyst precursor is formed by a process which comprises combining the sources of metal ions in aqueous solutions or aqueous mixtures, drying the resulting aqueous mixture to recover catalyst precursor, in dry particulate form

14. The composition of claim 13 wherein the aqueous mixtures are reacted at temperatures below about 100° C. and ambient, or near ambient, pressure.

15. The solid composition of claim 12 wherein the conditions of calcination comprise heating of the dry catalyst precursor, continuously or intermittently, from a preselected initial elevated temperature which is in a range upward from 250° C. to 400° C. to a 2^{nd} elevated temperature which is in the range of from 550° C. to 700° C., and ambient, or near ambient, pressure.

16. A solid composition that exhibits improved catalytic activity for ammoxidation or oxidation of propane and iso-butane in the gaseous phase, which composition is formed by a process comprising:
Providing a catalyst precursor, in dry particulate form, which comprises sources of metal ions in amounts consistent with a nominal mixed oxide material represented by the empirical formula:
MoVₐSb_{b}Nb_{c}Ti_{d}O_{δ}
where 0.1<a<1.0,
0.01<b<1.0,
0.01<c<0.25,
0.005<d<0.4, and
δ is the number of oxygen atoms required to maintain electro-neutrality of the other component elements present;
Calcining the catalyst precursor under conditions of calcination preselected such that at least one crystalline phase is formed in the resulting base catalyst which phase exhibits catalytic activity for ammoxidation or oxidation of propane and iso-butane in the gaseous phase;
Impregnating the base catalyst with an aqueous medium comprising sources of one or more element selected from the group consisting of antimony (Sb), cerium (Ce), niobium (Nb), tellurium (Te), and titanium (Ti) and thereafter drying the resulting material; and
Subjecting the dried material to heat treatment, under a gaseous atmosphere that is substantially free of dioxygen, at elevated temperatures of at least 400° C., thereby obtaining a mixed metal oxide composition that exhibits improved catalytic activity, for ammoxidation or oxidation of propane and iso-butane in the gaseous phase, relative to that of the base catalyst.

17. A process for catalytic conversion of propane or isobutane to an unsaturated nitrile or unsaturated carboxylic acid by ammoxidation in the presence of ammonia and a source of dioxygen or oxidation in the presence of a source of dioxygen, respectively, using a particulate solid catalyst which comprises one or more mixed metal crystalline oxide composition according to any proceeding claim.
